(19) United States
(12) Patent Application Publication
(10) Pub. No.: US 2013/0064712 A1
(43) Pub. Date: Mar. 14, 2013

(54) Composite comprising at least one type of perfluoroalkyl-perfluoro-phthalocyanine

(71) Applicants: Beate Roder, Falkensee (DE); Sergiu M. Gorun, Montclair, NJ (US); Robert Gerdes, Ulm (DE); Christian Litwinski, Berlin (DE)

(72) Inventors: Beate Roder, Falkensee (DE); Sergiu M. Gorun, Montclair, NJ (US); Robert Gerdes, Ulm (DE); Christian Litwinski, Berlin (DE)

(21) Appl. No.: 13/501,952

(22) PCT Filed: Oct. 12, 2010
(86) PCT No.: PCT/EP2010/006221

§ 371 Date: Nov. 28, 2012

Related U.S. Application Data
(60) Provisional application No. 61/251,516, filed on Oct. 14, 2009.

(51) Int. Cl. C08K 13/02; A61L 2/20; C01B 13/02

(52) U.S. Cl. 422/29; 106/287.18; 204/157.5

(57) ABSTRACT

The present invention relates to a composite comprising at least one type of perfluoroalkyl-perfluoro-phthalocyanine, and to a method of producing such composite. The present invention also relates to a method of generating singlet oxygen, a method of killing eukaryotic or prokaryotic cells and a method of sterilization, cleaning and/or decontamination. Moreover, the present invention relates to a composite or a device for use in a method of sterilization, cleaning and/or decontamination.

The present invention relates to a composite comprising at least one type of perfluoroalkyl-perfluoro-phthalocyanine, and to a method of producing such composite. The present invention also relates to a method of generating singlet oxygen, a method of killing eukaryotic or prokaryotic cells and a method of sterilization, cleaning and/or decontamination. Moreover, the present invention relates to a composite or a device for use in a method of sterilization, cleaning and/or decontamination.

Oxygen is one of the most important elements on Earth (Lane N (2002) Oxygen—The molecule that made the world (Oxford University Press, Oxford)). The majority of living organisms utilize oxygen for respiration and energy conversion. Singlet oxygen [¹O₂], the lowest electronic excited state of molecular oxygen, is a highly reactive species and can react very quickly and efficiently with a variety of chemical and organic molecules, thus playing a key role in photosensitized reactions in chemical and biological systems. ((a)Schweitzer C, Schmidt R (2003) Physical Mechanisms of Generation and Deactivation of Singlet Oxygen. Chem. Rev. 103:1685-1757 b)Fisher A M R, Murphree A L, Gomer C J (1995) Lasers Surg. Med. 17:2-31 c)Maisch T, Baier J, Franz B, Maier M, Landthaler M, Szeimies R M, Bäumler W (2007) The role of singlet oxygen and oxygen concentration in photodynamic inactivation of bacteria. Proc. Natl. Acad. Sci. U.S.A. 17:7223-7228 d) Niedre M J, Patterson M S, Wilson B C (2005) Singlet oxygen luminescence as an in vivo photodynamic therapy dose metric: validation in normal mouse skin with topical amino-levulinic acid. Br. J. Cancer 92:298-304)

Photodynamic therapy [PDT] makes use of the so-called photodynamic effect in which ¹O₂ is generated in the target tissue via energy transfer from the first excited triplet state of a photo-sensitizer to molecular oxygen in its triplet ground state. Today it is well known that ¹O₂ plays a key role in both the apoptotic and necrotic pathways of cell death induced by the photodynamic effect (a)Röder B (2000) in Encyclopedia Analytical Chemistry ed Meyers R A (Wiley, Chinchester) pp 302-320 b)Redmond R W, Kochevar I E (2006) Spatially Resolved Cellular Responses to Singlet Oxygen. Photochem. Photobiol. 82:1178-1186).

Recent studies have shown that the photodynamic effect can be used in the selective inactivation of microorganisms becoming a potential alternative for the treatment and eradication of microbial infections. The problem of inactivation of pathogenic bacteria causing various diseases of humans has become very relevant during recent years. Many strains are, or could become resistant to commonly applied disinfection methods and antibiotics. Therefore, there is a continuous and urgent need to search for alternative methods to which bacteria will not easily develop resistance. Examples for this new strategy are the use of bacteriophages (K. E. Cerveny, A. DePaola, D. H. Duckworth, P. A. Gulig: Phage therapy of local and systemic disease caused by Vibrio vulnificus in iron-dextran-treated mice, Infect. Immun. (2002) 70, 6251-6262) or synthetic antimicrobial peptides (U. S. Saijan, L. T. Tran, N. Sole, C. Rovaldi, A. Akiyama, P. M. Friden, J. F. Forstner, D. M. Rothstein: P-113D, antimicrobial peptide active against Pseudomonas aeruginosa, retains activity in the presence of sputum from cystic fibrosis patients, Agents Chemother. (2001) 45, 3437-3444). One of the most promising and innovative methods in this context is the photodynamic inactivation (PDI). This method uses a nontoxic dye, a photosensitizer (PS), activated by visible light to generate singlet oxygen and free radicals causing the death of microbial cells. Currently, the major use of this approach is in disinfection of blood products. Some attempts have been made to use it as an alternative method for food decontamination (L. Brovko, N. A. Romanova, Ch. Leslie, H. Ollivier, M. W. Griffiths: Photodynamic treatment for surface sanitation, Proc. SPIE, Vol. 5969, 596914 (2005); DOI:10.1117/12.628596; b) in: Progress in biomedical optics and imaging. ISSN 1605-7422, 2005, vol. 6, n 39, (SF6053—Antimicrobial photodynamic treatment for surface sanitation, project sponsored by the Ministry of Agriculture, Canada)). It is known that Gram (−)-bacteria are resistant against many photosensitizers that will cause easily phototoxicity in Gram (+)-bacteria (M. R. Hamblin, T. Hasan: Photodynamic therapy: a new antimicrobial approach to infectious disease? Photochem. Photobiol. Sci. (2004) 3, 436-450). It is also known that PS having a negative charge e.g. (G. P. Tegos, T. N. Demidova, D. Arcila-Lopez, H. Lee, T. Wharton, H. Gali, M. R. Hamblin: Cationic Fullerenes Are Effective and Selective Antimicrobial Photosensitizers, Chem. & Biol. (2005) 12, 1127-1135) or increasing the permeability of outer membranes are able to cause photodynamic damage also to Gram (−)-bacteria (M. R. Hamblin, T. Hasan: Photodynamic therapy: a new antimicrobial approach to infectious disease? Photochem. Photobiol. Sci. (2004) 3, 436-450). However several reports exist on successful killing of different antibiotic resistant bacteria, e.g. Staphylococcus aureus (a) M. Wainwright, D. A. Phoenix, S. L. Laycock, D. R. Wareing, P. A. Wright: Photobactericidal activity of phenothiazinium dyes against methicillin-resistant strains of Staphylococcus aureus, FEMS Microbiol. Lett. (1998) 160, 177-181 b) M. Wilson, C. Yianni: Killing of methicillin-resistant Staphyloccocuc aureus by low-power laser light, J. Med. Microbiol. (1995) 42, 62-66). Also, the successful use of antibacterial PDI combined with other methods is reported (R. E. Baddour, F. N. Dadami, M. C. Kolios, St. K. Bisland: High-Frequency Ultra-sound Assessment of Antimicrobial photodynamic Therapy In Vitro, J. Biol. Phys. (2007) 33, 61-66). The field of antibacterial PDI is relatively new, but recent reports indicate the use of PDI to treat infections in animal models (M. R. Hamblin, T. Hasan: Photodynamic therapy: a new antimicrobial approach to infectious disease? Photochem. Photobiol. Sci. (2004) 3, 436-450). In addition, a preliminary clinical study on efficient PDI treatment of ten patients with the diagnosis of aggressive periodentitis has been performed using phenothiazine as a PS. After three months a significant reduction of plaque scores in the same range as for conventional treatment (R. R. de Oliviera, H. O. Schwartz-Filho, A. B. Novaes Jr., M. Taba Jr.: Antimicrobial Photodynamic Therapy in the Non-Surgical Treatment of Aggressive Periodontitis: A Preliminary Randomized Controlled Clinical Study, J. Periodontol. (2007) 78, 965-973) was found. Further enhancement of antibacterial PDI was observed using “targeted nanoplatforms” (P. A. Suci, Z. Varpness, E. Gillitzer, T. Douglas, M. Young; Targeting and Photodynamic Killing of a Microbial Pathogen Using Protein Cage Architecture Functionalized with a Photosensitizer, Langmuir (2007) 23, 12280-12286). This strategy, however, requires a lot of biochemical and complicated gene-technical work. For this reason it is not suitable for every-day surface sanitation.

Possible future applications of antimicrobial PDI are the treatment of infections in wounds and surface infections of the cornea and skin. Antimicrobial PDI should be also applicable to clinical surface disinfection.

The broad area of antimicrobial PDI has been reviewed recently (M. R. Hamblin, T. Hasan: Photodynamic therapy: a new antimicrobial approach to infectious disease? Photochem. Photobiol. Sci. (2004) 3, 436-450). The literature data show that both Type I (radical formation) and Type II (singlet oxygen formation) photosensitization may cause antibacterial effects. Besides other compounds, tetrapyrroles [(M. R. Hamblin, T. Hasan: Photodynamic therapy: a new antimicrobial approach to infectious disease? Photochem. Photobiol. Sci. (2004) 3, 436-450) and references therein], with well defined properties may act as antimicrobial and antiviral photosensitizers. PSs penetration inside bacteria, followed by accumulation, is the conventional reaction pathway, discussed in the literature. The penetration step, especially for Gram (−)-bacteria, however, requires specially tailored molecules that ultimately enable intracellular accumulation.

In contrast to this “common” application of PDI against microbial organisms the use of PSs that do not accumulate in cells was discussed only recently (a) A. Ulatowska-Jarza, I. Holowacz, J. Razik, A. Wieliczko, K. Nowak, W. Strek, L. Czernielewski, H. Podbielska, New method for decontamination based on photodynamic activity—preliminary in vitro study on Gram-negative bacteria, Proc. Symp. Photon. Techn. For 7^{th} Framework Program, 12.-14. October 2006, Wroclaw, pp. 546-549 b) Mosinger, O. Jirsák, P. Kubat, K. Lang, B. Mosinger Jr.: Bactericidal nanofabrics based on photoproduction of singlet oxygen, J. Mater. Chem. (2007) 17, 164-166). Ulatowska-Jarza et al. applied several PSs in solution to bacteria cultures and investigated their phototoxicity against different Gram (−)-bacterial strains in culture (E. coli, P. aeruginosa). They found a good antimicrobial photodynamic action of the commercial available drug Photolon (a chlorine derivative). It should be mentioned, however, that the experimental conditions were not clearly shown and thus it is difficult to evaluate the validity of the results.

Mosinger et al. performed similar experiments using a more creative nano-technological approach for photosensitized singlet oxygen generation. They report the production of networks of polymer nanofibers (average diameter: 460 nm) using an industrial-scale electrospinning method. Polyurethane and tetraphenylporphyrin (as PS) were mixed in a DMF solution. As a result, nanofibers with immobilized PSs were produced. Adding some sodium dodecyl sulfate (SDS) they obtained a hydrophilic type of a nanofiber net. This system showed a high phototoxicity against E. coli bacteria. In addition to the simple counting of colony forming units (CFU) they used E. coli strain with DH5a with plasmid pGEM11Z that together produce b-galactosidase. Including X-gal as a b-galactosidase substrate in the agar the b-galactosidase producing bacterial colonies turned blue-green after transforming the X-gal substrate into an indole dye and thus becoming clearly visible.

However, the above research is unlikely to result in useful, practical applications The reason for this are the following facts:
the relatively low photostability of the PSs (TPP (tetraphenylporphyrin) and Photolon (a commercially available complex of chlorine e6 and polyvinylpyrrolidone))
their undesired ability to enter human cells thus causing phototoxic reactions
their tendency to aggregate thus preventing high monomeric PS concentrations in matrices.

Accordingly, it was an object of the present invention to provide for photosensitizer systems which are sufficiently stable. It was also an object of the present invention to provide for photosensitizer systems which have a high singlet oxygen quantum yield. Moreover, it was an object of the present invention to provide for photosensitizer system which do not aggregate.

All these objects are solved by a composite comprising

a) at least one type of perfluoroalkyl-perfluoro-phthalocyanine,

b) a solid matrix,

wherein said perfluoroalkyl-perfluoro-phthalocyanine is associated with said solid matrix and wherein said composite does not comprise any solvent.

In one embodiment, said perfluoroalkyl-perfluoro-phthalocyanine has one of the following structures:

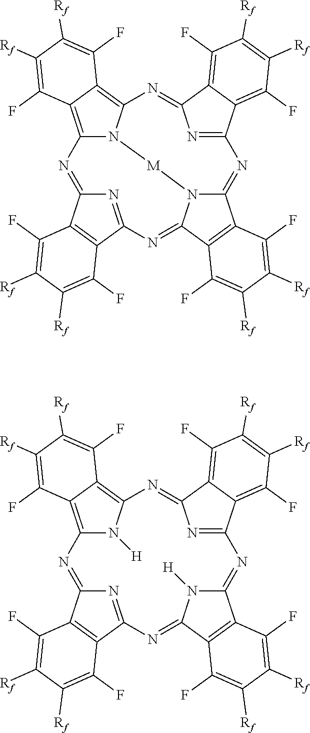

wherein M is a metal ion selected from transition series metals or rare earth series metals, and wherein Rf is a perfluoroalkyl group.

In one embodiment, said perfluoroalkyl group is selected from perfluoroisopropyl, perfluorohexyl, perfluorooctyl and combinations thereof.

In one embodiment, said perfluoroalkyl-perfluoro-phthalocyanine is selected from F₆₄PcH₂, and F₆₄PcZn.

In one embodiment, said solid matrix is an inorganic matrix or an organic polymeric matrix.

In one embodiment, said inorganic matrix is a nanoparticulate matrix.

In one embodiment, said inorganic matrix is made of a material selected from silicon-based materials, such as quartz, glass, silicon nitride, elemental silicon, including doped silicon and crystalline silicon, semiconductor materials, such as germanium, compound semiconductors, such as gallium arsenide, indium phosphide, aluminium-based materials, such as alumina, spinel, sapphire, ceramics, such as zirconia, fluoropolymers, such as Teflon®.

In one embodiment, said organic polymeric matrix is made of a material selected from a group comprising carbon-based polymers, such as polypropylene, polyethylene, silicon-based polymers, such as copolymers of polydimethylsiloxane and urea. As an example, such silicon-based polymers can be commercially obtained from Wacker (“Wacker films”, Geniomer®).

In one embodiment, said composite is a solid composite.

The objects of the present invention are also solved by a device comprising or being made, at least in parts, of said composite according to the present invention, wherein said device is a medical device, a surgical instrument, a patch or bandage for covering wounds.

The objects of the present invention are also solved by a method of producing a composite according to the present invention, comprising the steps:
a) providing, in any order, a matrix as defined above, and at least one type of perfluoroalkyl-perfluoro-phthalocyanine, as defined above,
b) exposing said matrix to said perfluoroalkyl-perfluoro-phthalocyanine, thereby associating said perfluoroalkyl-perfluoro-phthalocyanine with said matrix.

In one embodiment, said perfluoroalkyl-perfluoro-phthalocyanine is provided in a solvent as a solution in step a), and wherein step b) occurs by contacting said matrix with said solution, and subsequent removal of the solvent from said solution, e.g. by evaporation or drying.

The objects of the present invention are also solved by a method of generating singlet oxygen, comprising the steps:
a) providing a composite according to the present invention or a device according to the present invention,
b) irradiating said composite or said device by light, thereby generating singlet oxygen.

The objects of the present invention are also solved by a method of killing eukaryotic or prokaryotic cells, comprising the steps:
a) providing a composite according to the present invention or a device according to the present invention and bringing it into contact with eukaryotic or prokaryotic cells, or at a distance from said eukaryotic or prokaryotic cells of from 0 cm to 10 cm,
b) irradiating said composite or said device by light, and thus exposing said eukaryotic or prokaryotic cells to singlet oxygen.

The objects of the present invention are also solved by a method of sterilization, cleaning and/or decontamination, comprising the steps:
a) providing a composite according to the present invention or a device according to the present invention,
b) irradiating said composite or said device by light, thereby generating singlet oxygen.

The afore-mentioned methods are preferably performed in-vitro.

In one embodiment, the perfluoroalkyl-perfluoro-phthalocyanines are compounds having the structures as disclosed in WO 2006/086349, with or without central metal atom. In a preferred embodiment, the composite in accordance with the present invention does not comprise alpha-cyano-4-hydroxycinnamic acid. In one embodiment, the solid matrix is not a MALDI matrix, such as α-cyano-4-hydroxycinnamic acid, sinapic acid, 2-(4-hydroxyphenylazo)benzoic acid, 2, 5-dihydroxybencoic acid, 2, 4, 6-trihydroxyacetophenone, 3-hydroxypicolinic acid, 6-aza-2-thiothymine, T-2-(3-(4-t-butyl-phenyl)-2-methyl-2-propenylidene)malononitrile (DCTB).

If a metal ion is present in the phthalocyanine, it may be selected from the transition series metals or the rare earth series metals. Preferably, the metal ion is selected from zinc, cobalt or iron. In a particularly preferred embodiment, the perfluoroalkyl-perfluoro-phthalocyanine is selected from the group comprising F₆₄PcZn, and F₆₄PcH₂, with Fac denoting 1, 4, 8, 11, 15, 18, 22, 25-octafluoro-2, 3, 9, 10, 16, 17, 23, 24-octa-perfluoro-isopropyl-phthalocyanine, and Zn denoting zinc coordinated in the phthalocyanine. A person skilled in the art knows how to synthesize such perfluoroalkyl-perfluoro-phthalocyanine-compounds; ways of synthesizing are for example described in Lee et al., 2003, Chem. Commun., 1576-1577 and U.S. Pat. No. 6,511,971. The matrix in accordance with the present invention is a solid matrix. In one embodiment, it may be an inorganic matrix, in another embodiment, it may be an organic polymeric matrix. If the matrix is an inorganic matrix, it is, in one embodiment, a nanoparticulate matrix. The term “nanoparticulate”, as used herein, is meant to refer to a particulate structure of said matrix, wherein said matrix comprises particles, the average dimensions of which are in the range of from 1 nm to 900 nm.

In one embodiment, the composite in accordance with the present invention may also comprise more than one type of perfluoroalkyl-perfluoro-phthalocyanine.

In some embodiments, the solid matrix is an organic polymeric matrix, which is made of carbon-based polymers and/or silicon-based polymers These have the advantage that they may for example be prepared in the form of a film to which the perfluoroalkyl-perfluoro-phthalocyanine gets associated.

The “association” between the perfluoroalkyl-perfluoro-phthalocyanine and the solid matrix, in accordance with the present invention, may be based on a chemical linkage between the phthalocyanine compound and the matrix, or it may be a physical linkage, such as through van-der-Waals-interactions, or hydrophobic interactions. Both possibilities are envisaged within the present invention and are encompassed by the term “associated” as used herein.

The present invention also encompasses devices which comprise or are made, either entirely or at least in parts, of the composite in accordance with the present invention. Preferably such devices are medical devices or surgical instruments where there is a need for maintenance of a clean sterile environment. The same also applies to patches or bandages for covering wounds which may also comprise the composite in accordance with the present invention.

The present invention also considers methods of producing a composite in accordance with the present invention, in which a) a matrix as defined above and at least one type of perfluoroalkyl-perfluoro-phthalocyanine as defined above, are provided in any order, and b) thereafter said matrix is exposed to said perfluoroalkyl-perfluoro-phthalocyanine, whereby the perfluoroalkyl-perfluoro-phthalocyanine becomes associates with said matrix. Preferably, in such a method of production, the perfluoroalkyl-perfluoro-phthalocyanine is provided in step a) as a solution comprising a solvent and said perfluoroalkyl-perfluoro-phthalocyanine compound, and wherein step b) occurs by contacting said matrix with said solution. Thereafter, the solvent is removed from said solution, for example by evaporation or drying. The “contacting” step just mentioned may occur by dipping the matrix into said solution of perfluoroalkyl-perfluoro-phthalocyanine or by dropping said solution onto said matrix or by imbibing said matrix into said solution, or by soaking said matrix with said solution. It should be noted, however, that, in accordance with the present invention, the composite does not comprise any solvent, i.e. it is dry and the perfluoroalkyl-perfluoro-phthalocyanine is associated with said solid matrix.

The present invention also relates to a method of generating singlet oxygen and to a method of sterilization, cleaning and/or decontamination, comprising the steps:

a) providing a composite according to the present invention or a device according to the present invention, and

b) irradiating said composite or said device by light, thereby generating singlet oxygen. The term “irradiating by light”, as used herein is meant to refer to an irradiation applying a wavelength range or a subset thereof from 300 nm to 800 nm. In a more preferred embodiment, the wavelength range is from 350 nm to 750 nm. In yet another embodiment, the wavelength range is from 600 nm to 720 nm, preferably 650 nm to 700 nm. In yet a further embodiment, the wavelength range is from 670 nm to 690 nm. In one embodiment, “irradiating” means “exposing to daylight or sunlight”, or “exposing to a light mimicking the solar spectrum or a part thereof”.

The present invention also relates to a method of killing eukaryotic or prokaryotic cells, comprising the steps a) providing a composite or a device according to the present invention and bringing it into contact with eukaryotic or prokaryotic cells to be killed or at a distance of from 0 cm to 10 cm of said eukaryotic or prokaryotic cells,

b) irradiating said composite or said device by light, as outlined above, thereby generating singlet oxygen, and thus exposing said cells to the generated singlet oxygen.

The present invention also relates to a pharmaceutical composition comprising the composite in accordance with the present invention.

The present invention also relates to the composite in accordance with the present invention for use in a method of treating an infection in a patient, such as a bacterial infection or a viral infection, or a disease, wherein the elimination of cells is desired, said method comprising the administration of said composite to said patient and subsequently irradiating said composite by light, in the aforementioned sense.

Moreover, the present invention also relates to a method of photoinactivating infectious agents, such as viruses or bacteria, in a body fluid in-vitro, said method comprising contacting the body fluid in-vitro with said composite and irradiating said composite by light.

As outlined above, the irradiation may also simply occur by exposing the composite to natural light, such as sunlight.

The afore-mentioned methods are preferably performed in-vitro.

The present inventors have surprisingly found that composites comprising perfluoroalkyl-perfluoro-phthalocyanine and a solid matrix are much more stable than the photosensitizer systems known so far. The composites in accordance with the present invention have a high singlet oxygen quantum yield, and they have no tendency to accumulate. Without wishing to be bound by any mechanistic explanation, the latter aspect is likely to be due to the chemical structure caused by the bulky fluorinated groups. The composites in accordance with the present invention are versatile and can be used in a wide range of applications, including sterilization, decontamination and cleaning.

In the following, reference is made to the figures, wherein

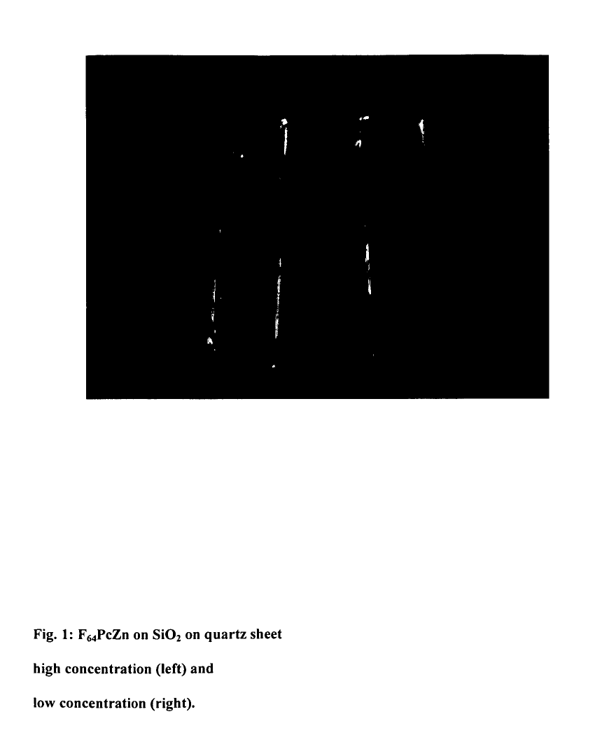

FIG. 1 shows F₆₄PcZn on SiO₂ on quartz sheet,

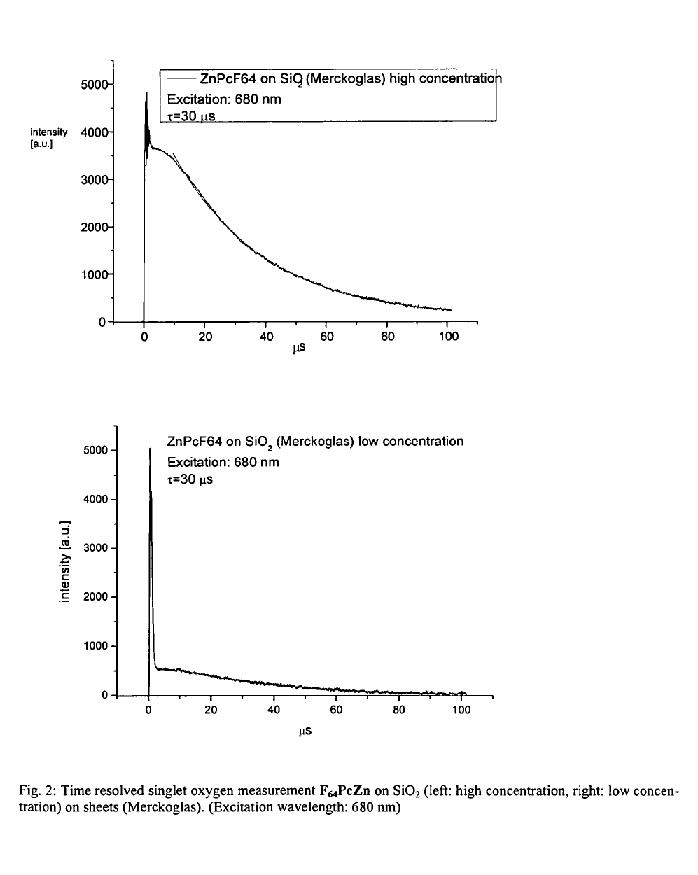

FIG. 2 shows time resolved singlet oxygen measurements with F₆₄PcZn on SiO₂ (left panel: high concentration, right panel: low concentration on sheets of Merckoglass® Si-containing carbon-based polymer. (Excitation wavelength: 680 nm);

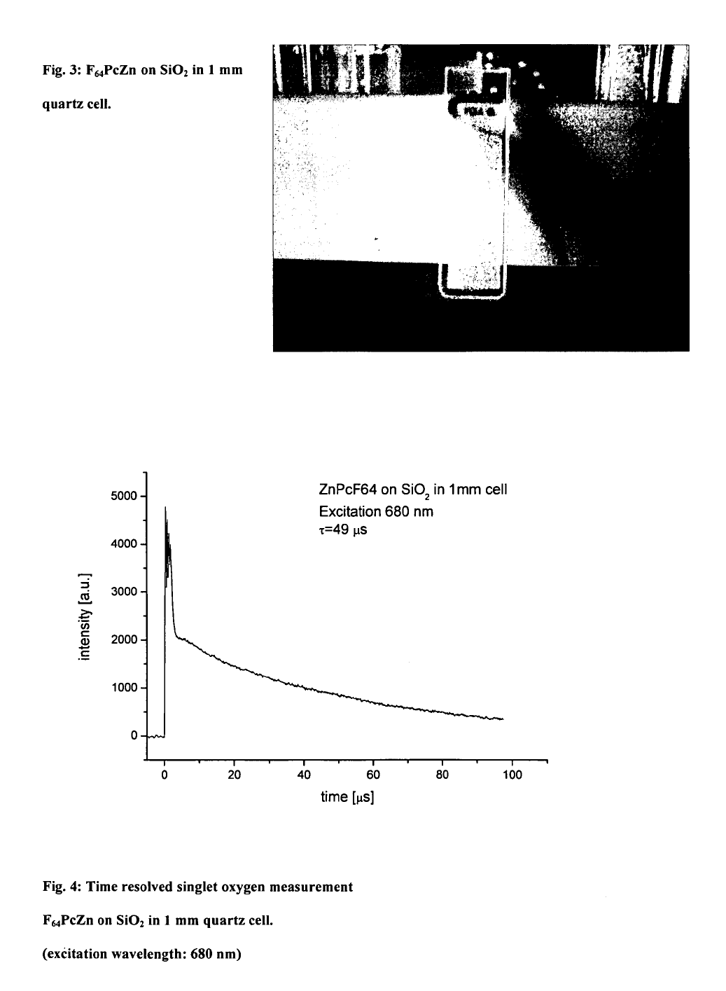

FIG. 3 shows a photograph of F₆₄PcZn on SiO₂ in 1 mm quartz cell;

FIG. 4 shows a time resolved singlet oxygen measurement of F₆₄PcZn on SiO₂ in 1 mm quartz cell (excitation wavelength: 680 nm);

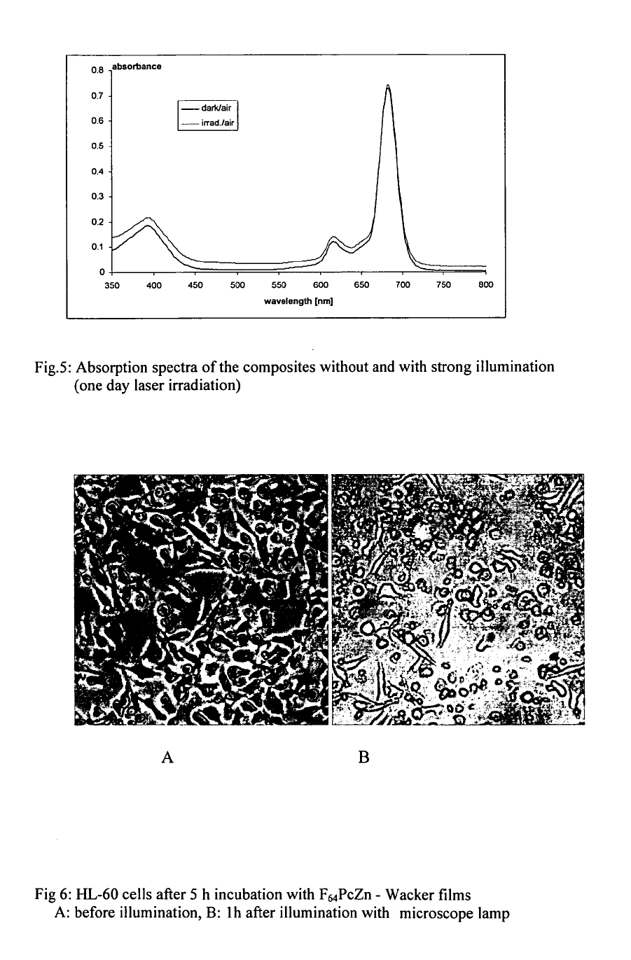

FIG. 5 shows absorption spectra of the composites in accordance with one embodiment of the present invention without and with strong illumination;

FIG. 6 shows HL-60 cells after 5 h incubation with F₆₄PcZn-Wacker-films; panel A: before illumination, panel B: 1 h after illumination with a microscope lamp;

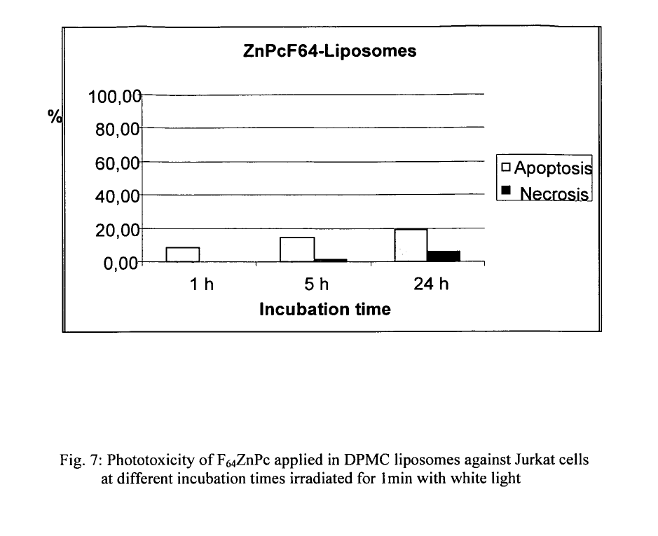

FIG. 7 shows the phototoxicity of F₆₄PcZn applied in dimyristoylphosphatidylcholin (DMPC) liposomes against Jurkat cells at different incubation times (1 h, 5 h and 24 h) irradiated for 1 min with white light.

Moreover, reference is made to the following examples, which are given to illustrate the invention not to limit the same.

### EXAMPLES

### Example 1

### Chemical Structure of Molecules and Synthesis

The molecules have a special chemical nature, as mentioned briefly above, see also FIG. 1. They exhibit a large macrocyclic ring with extended, aromatic conjugation that usually renders them planar. However, the C—H bonds normally present in aromatic hydrocarbons are totally eliminated and replaced by either C—F or C—C bonds. The terminal carbon atoms however, are not capped by H but by F, thus forming stable aliphatic groups. Unlike aromatic C—H bonds, the C—F bonds are very stable from a thermal and chemical point of view. Lastly, the 3-dimensional nature of the perfluoroalkyl groups (branched as opposed to linear) imparts anti-stacking properties to the phthalocyanines to which they are attached. This property favors the formation of the singlet oxygen since it prevents sensitizer deactivation via stacking interactions. The synthesis of the perfluoroalkyl-perfluoro-phthalocyanines can e.g. be performed as described in Lee et al., 2003, Chem. Commun., 1576-1577 and U.S. Pat. No. 6,511,971.

### Example 2

### Preparation of Composites and Demonstration of Singlet Oxygen Generation

F₆₄PcZn was deposited on silicagel SiO₂. The procedure involves the dissolution of the phthalocyanine in an organic solvent in a glass vessel, introduction of the silicagel and evaporation of the solvent. The resulting composite is abbreviated F₆₄PcZn∈SiO₂.

a) Experiment I: F₆₄PcZn on SiO₂ deposit on sheets with Merckoglas©

Sample description: deposit fluid Merckoglas© and F₆₄PcZn on SiO₂ on quartz sheet
drying 15 minutes

Time resolved measurements of simplet oxygen luminescence, as shown in FIG. 2, gave the following results:
Singlet oxygen decay time of F₆₄PcZn on SiO₂ (Merckoglas): 30 μs
First peak (up to 3 μs) results from the scattering signal of sample

b) Experiment II: F₆₄PcZn on SiO₂ in 1 mm quartz cell

Preparation of sample: F64PcZn

on SiO₂ filled in 1 mm quartz cell (see FIG. 3)

Time resolved measurement of singlet oxygen luminescence of Experiment II can be seen in FIG. 4:

The results of the experiment of FIG. 4 are:
Singlet oxygen decay time of F₆₄PcZn on SiO₂ in 1 mm quartz cell 49 μs
First peak (up to 4 μs) results from the scattering signal of sample, broader scattering signal as with Merckoglas
Lifetime is 19 μs longer than with Merckoglas, less quenching than in Merckoglas

The results clearly show a very long lifetime of singlet oxygen in air. Together with the very high singlet oxygen quantum yield of the ZnPcF64 of around 56% the composite material could be used for efficient photosensitization in solid-state, as opposed to solution.

The results also show that no aggregation occurs in the polymers. This is caused by the chemical structure of the molecule.

### Example 3

### Stability-Test for F₆₄PcZn on SiO₂ (35-60 mesh) Under Air

F₆₄PcZn was deposited on two samples of SiO₂, about 0.100 g of material each.

Sample 1, was kept in the dark, but in contact with atmosphere. This sample was labelled “dark/air”.

Sample 2 was irradiated in air for two hours with two lamps (100 mW/cm²). The light intensity was similar to that of a sunny day. This sample was labelled “irrad./air”.

At the end of the experiments (two hours) the materials were washed with 10 ml of ethanol which removed the colour, i.e. F₆₄PcZn. The samples were diluted 1:2 with ethanol and the UV-Vis was recorded, see the Figure below.

The Q-band absorption bands at ˜670 nm are virtually the same, with no broadening, shift or shoulders, while the general appearance of the spectra originating from the irradiated and dark samples is maintained. There is no evidence of decomposition products or aggregation, although the baseline absorptions appear slightly different.

### Example 4

### Demonstration of the Phototoxicity of F₆₄PcZn Loaded Wacker Films Upon HL-60 Macrophages

Loading of the Wacker Films
cutting the foil in small pieces (0.5 cm×0.5 cm)
dipping the pieces in to 70% Ethanol to decontaminate them and drying by air under the sterile bench
dropping 30 μl Fluor-Pc solution (ethanol) to every piece and drying by air

Cell Culture and Incubation

Medium: RPMI with 10% FCS, 4 mM L-glutamine, 100 U/ml of penicillin/streptomycin Incubation conditions: 37° C., 5% CO2, 100% humidity

Because of the high hydrophobicity of the Wacker films it was impossible to grow the cells directly on the films.

The promyelocytic leukemia cell line HL-60 is a suspension cell line. After treatment with 10 nM PMA (phorbol myristate acetate) the cells differentiate within 24 h to adherent macrophage like cells.

Differentiation of HL-60 cells take place on a 24-well plate. 1 ml per well with ca. 500 000 cells/ml, 24 h. After differentiation the medium containing some not differentiated suspension cells has to be removed. One piece of the Wacker foil was put per well on the cell layer and 200 μl fresh medium containing 10 nM PMA was added. Then the cells were kept in darkness for 5 h.

Illumination and Phototoxicity

Observation of the cells with an inverse light microscope with low light intensity (as low as possible) to check the dark toxicity.

Illumination of the cells with the full power of microscope lamp for 30 sec each well.

Additional incubation for 1 h and observation of the phototoxicity

Results of this experiment can be seen in FIG. 6.

Because the high hydrophobicity of the films and the mechanical stress the growing of the adherent cells is not ideal. Nevertheless, the phototoxicity of the F₆₄PcZn—Wacker films is detectable (see FIG. 6). About 70% of the illuminated cells are showing strong morphological changes looking like a mixture of necrotic and apoptotic effects.

### Example 5

### Phototoxicity of Free F₆₄PcZn Against Human Cells

If the composite is used also on human skin there is a probability of PS loss by the photoactive foil. In order to protect the human body against undesired phototoxic effects it is important to make sure that the free molecules are not able to enter human cells.

In order to demonstrate lack of cell penetration for our molecules the inventors used Jurkat cells that are known to accumulate tetrapyrrolic PS very well and in a high amount (a) Paul A, Hackbarth S, Mölich A, Seifert M, Röder B (2003) Comparative Study on the Photosensitization of Jurkat Cells in vitro by Pheophorbide-a and a Pheophorbide-a Diaminobutane poly-propylene-imine Dendrimer Complex. Laser Phys. 13:22-29 b) M. Regehly , K. Greish, F. Rancan, H. Maeda, B. Röder: Water-soluble polymer conjugates of ZnPP for photodynamic therapy, Bioconjugate Chemistry, 18 (2007) 494-499. c) K. Chen, A. Preuβ, S. Hackbarth, M. Wacker, K. Langer, B. Röder: Novel photosensitizer-protein nanoparticles for Photodynamic Therapy: Photophysical characterization and in vitro investigations, J. Photochem. Photobiol.: B, in press May 2009). It is important to mention that phthalocyanines also belong to the class of tetrapyroles and thus the above comparison is meaningful. Thus, incubating the cell suspensions according to the procedure described in a) Paul A, Hackbarth S, Mölich A, Seifert M, Röder B (2003) Comparative Study on the Photosensitization of Jurkat Cells in vitro by Pheophorbide-a and a Pheophorbide-a Diaminobutane polypropylene-imine Dendrimer Complex. Laser Phys. 13:22-29 b) M. Regehly, K. Greish, F. Rancan, H Maeda, B. Röder: Water-soluble polymer conjugates of ZnPP for photodynamic therapy, Bioconjugate Chemistry, 18 (2007) 494-499. c) K Chen, A. Preuβ, S. Hackbarth, M. Wacker, K Langer, B. Röder: Novel photosensitizer-protein nanoparticles for Photodynamic Therapy: Photophysical characterization and in vitro investigations, J. Photochem. Photobiol.: B, in press May 2009 with F₆₄PcZn resulted in undetectable accumulation of the dye inside the cells.

To evaluate the possible accumulation under worst case scenario the inventors have used dimyristoylphosphatidylcholine (DMPC) liposomes as carriers for the PS. This procedure simulated the possible formation of lipid vesicles during applications on skin of some crème or oily drug suspensions which might incorporate the F₆₄ZnPc.

Phototoxicity of F₆₄ZnPc-Liposomes
0.75 ml ZnPcF64/liposomes (DPMC) in 2 ml H₂O
concentration of F₆₄ZnPc in the cell medium 7 μM
irradiation: 1 min (broad band illumination)
   FIG. 7 demonstrates that the application in liposomes did not result in significant accumulation as reflected by less than 20% phototoxicity for one cellular replication cycle.

### Example 6

### Photodynamic Inactivation of Gram(−) Bacteria

This work uses SURE® 2 Supercompetent Cells from STRATAGENE (commercial supplier). This is a non-pathogenic E. coli strain sensitive to redox-stress and resistant to kanamycine, tetracycline, and chloramphenicol antibiotics. The selection of this strain is based on our prior experience in using these cells for phototoxic studies in Berlin.

Methodology: The bacterial colonies are irradiated using a broadband illumination source (white LED). After irradiation the plates are incubated for several hours (depending on the cells under investigation up to 24 h) at 37° C. After that the colonies on the plates are counted and colony forming units (CFU) calculated.

The composites in accordance with the present invention provide for stable and highly active solid-state photosensitizers which open up the possibility of constructing materials with self-cleaning and/or self-sterilizing properties. This is of importance in surgical instruments and medical devices that come in contact with the human body, such as sterile space coatings, heterogeneous catalysts and environmentally benign cleaning agents. Furthermore, the composites in accordance with the present invention can also be used in patches and bandages that may be brought in contact with the human body so as to maintain a sterile environment and/or reduce bacterial/viral contaminations. This may for example be important for the treatment of superficial wounds. The composites of the present invention are also useful in methods of inactivating infectious agents in body fluids, such as blood.

The features of the present invention disclosed in the specification, the claims and/or in the accompanying drawings, may, both separately, and in any combination thereof, be material for realizing the invention in various forms thereof.

1. A composite comprising
a) at least one type of perfluoroalkyl-perfluoro-phthalocyanine, and
b) a solid matrix,
wherein said perfluoroalkyl-perfluoro-phthalocyanine is associated with said solid matrix and wherein said composite does not comprise any solvent.

2. The composite according to claim 1, wherein said perfluoroalkyl-perfluoro-phthalocyanine has one of the following structures: 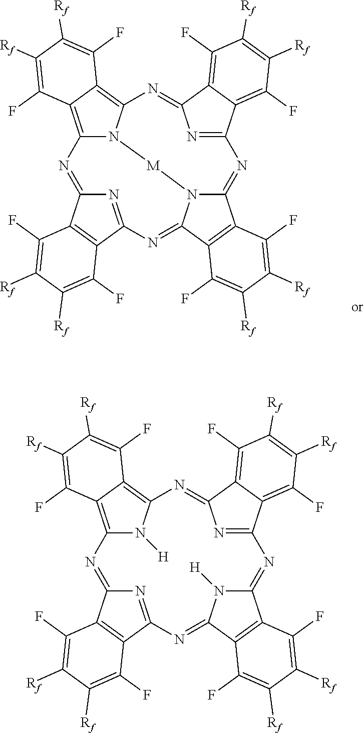
wherein M is a metal ion selected from transition series metals and rare earth series metals, and wherein Rf is a perfluoroalkyl group.

3. The composite according to claim 1, wherein said perfluoroalkyl group is selected from perfluoroisopropyl, perfluorohexyl, perfluorooctyl and combinations thereof.

4. The composite according to claim 1, wherein said perfluoroalkyl-perfluoro-phthalocyanine is selected from F₆₄PcH₂ and F₆₄PcZn.

5. The composite according to claim 1, wherein said solid matrix is an inorganic Matrix or an Organic polymeric matrix.

6. The composite according to claim 5, wherein said inorganic matrix is a nanoparticulate matrix.

7. The composite according to claim 5, wherein said inorganic matrix is made of a material selected from silicon-based materials semiconductor materials, compound semiconductors, indium phosphide, aluminium-based materials, spinel, sapphire, ceramics, and fluoropolymers.

8. The composite according to claim 5, wherein said organic polymeric matrix is made of a material selected from a group consisting of carbon-based polymers, silicon-based polymers, and urea.

9. The composite according to claim 1, wherein said composite is a solid composite.

10. A device comprising a composite comprising at least one type of perfluoroalkyl-perfluoro-phthalocyanine and a solid matrix, wherein said perfluoroalkyl-perfluoro-phthalocyanine is associated with said solid matrix and wherein said composite does not comprise any solvent, wherein said device is a medical device, a surgical instrument, a patch or a bandage for covering wounds.

11. A method of producing a composite of claim 1 wherein said method comprises the steps:
a) providing, in any order, a solid matrix, and at least one type of perfluoroalkyl-perfluoro-phthalocyanine, and
b) exposing said matrix to said perfluoroalkyl-perfluoro-phthalocyanine, thereby associating said perfluoroalkyl-perfluoro-phthalocyanine with said matrix.

12. The method according to claim 11, wherein said perfluoroalkyl-perfluoro-phthalocyanine is provided in a solvent as a solution in step a), and wherein step b) occurs by contacting said matrix with said solution, and subsequent removal of the solvent from said solution.

13. A method of generating singlet oxygen, comprising the steps:
a) providing a composite of claim 1, and
b) irradiating said composite by light, thereby generating singlet oxygen.

14. A method of generating singlet oxygen, comprising the steps:
c) providing a device according to claim 10, and
d) irradiating said device by light, thereby generating singlet oxygen.

15. A method of killing eukaryotic or prokaryotic cells, comprising the steps:
a) providing a composite of claim 1, and bringing it into contact with eukaryotic or prokaryotic cells, or at a distance from said eukaryotic or prokaryotic cells of from 0 cm to 10 cm, and
b) irradiating said composite by light, and thus exposing said eukaryotic or prokaryotic cells to singlet oxygen.

16. A method of killing eukaryotic or prokaryotic cells, comprising the steps:
e) providing a device according to claim 10 and bringing it into contact with eukaryotic or prokaryotic cells of from about 0 cm to about 10 cm, and
f) irradiating said device by light, and thus exposing said eukaryotic or prokaryotic cells to singlet oxygen.

17. A method of sterilization, cleaning and/or decontamination, comprising the steps:
a) providing a composite of claim 1, and
b) irradiating said composite by light, thereby generating singlet oxygen.

18. A method of sterilization, cleaning and/or decontamination, comprising the steps:
g) providing a device according to claim 10, and
h) irradiating said device by light, thereby generating singlet oxygen.
